(19) United States
(12) Patent Application Publication
(10) Pub. No.: US 2012/0058531 A1
(43) Pub. Date: Mar. 8, 2012

(54) CONTINUOUS CULTURE FOR 1,3-PROPANEDIOL PRODUCTION USING HIGH GLYCERINE CONCENTRATION

(75) Inventors: Michel Chateau, Riom (FR); Jean-Yves Dubois, Saint Beauzire (FR); Philippe Soucaille, Deyme (FR)

(73) Assignee: METABOLIC EXPLORER, Saint Beauzire (FR)

(21) Appl. No.: 13/318,237

(22) PCT Filed: May 5, 2010
(86) PCT No.: PCT/EP2010/056078

§ 371 Date: Oct. 31, 2011

Related U.S. Application Data
(60) Provisional application No. 61/175,564, filed on May 5, 2009.

(30) Foreign Application Priority Data
May 5, 2009 (EP) 09159401.0

(51) Int. Cl. C12P 7/18; C07C 31/20; C12N 1/14; C12N 1/16; C12N 1/36; C12N 1/20

(52) U.S. Cl. 435/158; 435/245; 435/252.7; 435/255.1; 435/254.1; 435/252.8; 435/252.5; 568/852

(57) ABSTRACT

The present invention concerns a new method for the production of 1,3-propanediol comprising culturing a microorganism on a culture medium with high glycerine content. The invention also concerns a new microorganism, or strain of microorganism, adapted for the production of 1,3-propanediol from medium comprising high glycerine content.

The invention also concerns an “adapted microorganism” which glycerol metabolism is directed to 1,3-propanediol production, and which is allowed to grow in the presence of a high concentration of industrial glycerine.

The invention also concerns a biosourced 1,3-propanediol obtained by the process thereof. Finally the invention concerns the use of the above described biosourced 1,3-propanediol as extender chain in thermoplastic polyurethane, as monomers in polytrimethylene terephtalate and as a component in cosmetics formulations.

The present invention concerns a new method for the production of 1,3-propanediol comprising culturing a microorganism on a culture medium with high glycerine content. The invention also concerns a new microorganism, or strain of microorganism, adapted for the production of 1,3-propanediol from a medium comprising high glycerine content. The invention also concerns an “adapted microorganism” which glycerol metabolism is directed to 1,3-propanediol production, and which is allowed to grow in the presence of a high concentration of industrial glycerine. The invention also concerns a biosourced 1,3-propanediol obtained by the process of the invention. Finally the invention concerns the use of the above described biosourced 1,3-propanediol as extender chain in thermoplastic polyurethane, as monomers in polytrimethylene terephtalate and as a component in cosmetics formulations.

### BACKGROUND OF THE INVENTION

1,3-Propanediol (PDO) is one of the oldest known fermentation products. It was reliably identified as early as 1881 by August Freund, in a glycerol-fermenting mixed culture obviously containing Clostridium pasteurianum as the active organism. Quantitative analysis of the fermentation of different enterobacteria producing PDO (trimethylene glycol, propylene glycol) started at the microbiology school of Delft as early as 1928 and was successfully continued at Ames, Iowa in the 1940s. In the 1960s, interest shifted to the glycerol-attacking enzymes, in particular to the glycerol and diol dehydratases, as these enzymes were peculiar in requiring coenzyme B12. PDO-forming clostridia were first described in 1983 as part of a process to obtain a specialty product from glycerol-excreting algae (Nakas et al., 1983). PDO is a typical product of glycerol fermentation and has not been found in anaerobic conversions of other organic substrates. Only very few organisms, all of them bacteria, are able to form it. They include enterobacteria of the genera Klebsiella (K. pneumoniae), Enterobacter (E. agglomerans) and Citrobacter (C. freundii), lactobacilli (L. brevis and L. buchneri) and clostridia of the C. butyricum and the C. pasteurianum group.

Analysis of the fermentation products shows that part of the glycerol is converted to the same products as in the sugar fermentation of this different species, namely acetic acid, 2,3-butanediol, butyric acid, lactic acid, ethanol and succinic acid. This conversion provides the necessary energy for growth but, for many of the products, reducing equivalents are also released, which are used in a reductive conversion of glycerol to PDO. Butyrate formation, which lowers the PDO yield in clostridia, is somewhat comparable to ethanol formation in Klebsiella, but it seems to be more dependent on growth rate. Butyrate decreases rapidly with the dilution rate, even in the absence of substrate excess. In any case, changes in the acetate/butyrate ratio do not have such a great impact on the PDO yield.

PDO, as a bi-functional organic compound, could potentially be used for many synthesis reactions, in particular as a monomer for polycondensations to produce polyesters, polyethers and polyurethanes. PDO can be produced by different chemicals routes but they generate waste streams containing extremely polluting substances and the cost of production is then high and chemically produced PDO could not compete with the petrochemically available, diols like 1,2-ethanediol, 1,2-propanediol, and 1,4-butanediol. Therefore, in the past PDO has only found niche applications of negligible market volume. It is the reason why in 1995 Dupont started a research program for the biological conversion of glucose to PDO. Although this process is environmentally friendly it has the disadvantage to i) use vitamin B12 a very expensive cofactor and ii) be a discontinuous process due to the instability of the producing strain. Due to the availability of large amount of glycerol issued from bio-diesel industry, a continuous process, vitamin B12 free with a higher carbon yield would be advantageous.

It is known in the art that PDO can be produced from glycerine, an unwanted by product of the biodiesel production which contains roughly 80-85% of glycerol mixed with salts and water. C. butyricum was previously described as been able to grow and produce PDO from industrial glycerol in batch and two-stage continuous fermentation (Papanikolaou et al., 2000). However, at the highest glycerol concentration, the maximal PDO titre obtained was 48.1 g/L at a dilution rate of 0.02 h⁻¹, meaning a productivity of 0.9 g/L/H. The cultures were conducted with a maximum glycerol concentration in the fed medium of 90 g/L and in the presence of yeast extract, a costly compound containing organic nitrogen that is known by the man skilled in the art to help in increasing bacterial biomass production.

WO2006/128381 discloses the use of this glycerine for the production of PDO with batch and Fed-Batch cultures using natural PDO producing organisms such as Klebsiella pneumoniae, C. butyricum or C. pasteuricum. Furthermore, the medium used in WO2006/128381 also contains yeast extract. As described in this patent application, the maximal productivity reached is comprised between 0.8 and 1.1 g.l.h⁻¹.

The performance of a C. acetobutylicum strain modified to contain the vitamin B12-independent glycerol-dehydratase and the PDO-dehydrogenase from C. butyricum, called C. acetobutylicum DG1 pSPD5 has been described in Gonzalez-Pajuelo et al., 2005. This strain originally grows and produces PDO with a fed medium containing up to 120 g.l⁻¹ of pure glycerol. In addition, analyses with a fed medium of containing a maximum 60 g.l⁻¹ of pure or industrial glycerol did not identify any differences. When comparing C. butyricum to C. acetobutylicum DG1 pSPD5 a global similar behaviour was observed by the authors Gonzalez-Pajuelo et al., 2006.

However, the same C. acetobutylicum DG1 pSPD5 strain tested with 105 g.l⁻¹ of industrial glycerol in a synthetic fed medium without organic nitrogen, could not give the same performances as with the same concentration of pure glycerol (see example 1).

The present invention provides means for the production of 1,3-propandeiol with high concentration of industrial glycerine and where a higher PDO titre and productivity can be reached.

### BRIEF DESCRIPTION OF THE INVENTION

The present invention concerns a method for the production of 1,3-propanediol in a continuous fermentation process of glycerine, comprising culturing a producing microorganism on a culture medium, said producing microorganism allowing the conversion of glycerol into 1,3-propanediol, and recovering the 1,3-propanediol, characterized in that the culture medium comprises a high concentration of industrial glycerine, said industrial glycerine comprising glycerol, and wherein the producing microorganism is a microorganism previously adapted to grow in the presence of a high concentration of industrial glycerine.

In preferred embodiments, the glycerol is present in the culture medium at a concentration comprised between 90 and 120 g/L, preferably a concentration of about 105 g/L.

The culture medium is preferably a synthetic medium, without addition of any organic nitrogen source, and in particular without yeast extract.

The industrial glycerine is particularly a by-product from biodiesel production.

The producing microorganism is preferably a bacteria, more preferably selected from members of the genus Clostridium, particularly Clostridium acetobutylicum.

The producing microorganism is advantageously a genetically modified microorganism to allow improved production of 1,3-propanediol from glycerol.

In a preferred embodiment, the glycerol dehydratase activity in the producing microorganism is independent of the presence of coenzyme B12 or one of its precursors and is derived from Clostridium butyricum.

Preferably, the producing microorganism is a microorganism previously adapted to grow on the culture medium having a high concentration of industrial glycerine by culturing a microorganism on a culture medium comprising a high concentration of industrial glycerine at a low dilution rate and selecting the adapted microorganism.

Eventually, the 1,3-propanediol is further purified.

The present invention also concerns a method for the modification of a microorganism into a microorganism adapted to grow in the presence of a high concentration of industrial glycerine, comprising culturing the microorganism on a culture medium comprising a high concentration of industrial glycerine at a low dilution rate and selecting the adapted microorganism able to grow on the culture medium having a high concentration of industrial glycerine.

The microorganism is advantageously cultured at low dilution rate over a period ranging from 24 hours to 10 days, preferably more than 2 days, more preferably about 8 days.

The dilution rate is generally comprised between 0.005 and 0.1 h⁻¹, preferably between 0.005 and 0.02 h⁻¹. The dilution rate can be changed during the adaptation method, eventually with a first step comprised between 0.005 and 0.02 h⁻¹ and a second step where the dilution rate is increased up to 0.1 h⁻¹, preferably 0.06 h⁻¹.

The present invention also concerns an adapted microorganism, a producing microorganism adapted to grow in the presence of a high concentration of industrial glycerine obtainable by the method as disclosed above and below.

The invention also concerns a biosourced 1,3-propanediol obtained by the method of the invention.

The invention also concerns a biosourced 1,3-propanediol characterized by a combination of ¹³C and ¹⁸O isotopic values selected among δ¹³C lower than −34% and δ¹⁸O between 21.9% and 0.5%, preferably δ¹³C is lower than −35% and δ¹⁸O is between 21.9% and 0.5%, more preferably δ¹³C is between −35,05% and −36.09% and δ¹⁸O is between 21.9% and 17.34%.

In a preferred embodiment, the biosourced 1,3-propanediol is characterized by a δ¹³C/δ¹⁸O isotopic ratio value comprised between −2 and 0, preferably between −1 and −0.2 and more preferably between −0.65 and −0.4.

The present invention also concerns the use of biosourced 1,3-propanediol obtained by the method of the invention, or as defined above, as an extender chain in thermoplastic polyurethane, as monomers in polytrimethylene terephtalate or as a component in cosmetic formulations.

### DETAILED DESCRIPTION OF THE INVENTION

The invention is related to a method for the production of 1,3-propanediol in a continuous fermentation process of glycerine, comprising culturing a producing microorganism in a culture medium, said producing microorganism allowing the conversion of glycerol into 1,3-propanediol, and recovering the 1,3-propanediol, characterized in that the culture medium comprises a high concentration of industrial glycerine, said industrial glycerine comprising glycerol, and wherein the producing microorganism is a microorganism previously adapted to grow in the presence of a high concentration of industrial glycerine.

The term “microorganism” means a microorganism selected among the group consisting of bacteria, yeast and fungi. Preferentially, the microorganism is a bacterium preferably selected among the group consisting of Enterobacteriaceae, Bacillaceae, Clostridiaceae, Streptomycetaceae and Corynebacteriaceae. More preferentially, the bacterium is selected among the group consisting of Escherichia sp. (preferably Escherichia coli), Klebsiella sp (preferably Klebsiella pneumoniae), Bacillus sp. (preferably Bacillus subtilis), Clostridium sp. (preferably Clostridium acetobutylicum and Clostridium butyricum) and Corynebacterium sp (preferably Corynebacterium glutamicum).

The terms “Escherichia”, “Klebsiella”, “Bacillus”, “Clostridium” and “Clostridia” and “Corynebacterium” refer to all kind of bacteria belonging to these families or genera.

The term “producing microorganism” or “producing strain of microorganism” means a microorganism or a strain of microorganism wherein the glycerol metabolism of the microorganism is directed to 1,3-propanediol production.

A “microorganism being adapted” means a microorganism being modified to be able to grow on high concentrations of industrial glycerine.

An “appropriate culture medium” or a “culture medium” refers to a culture medium optimized for the growth and the diol-production of the producing strain.

The terms “high glycerine content” or “high concentration of glycerine” means more than 90 g/l of glycerol in the culture medium. In preferred embodiments, the culture medium comprises glycerol at a concentration comprised between 90 and 120 g/L, preferably about 105 g/L.

“Industrial glycerine” means a glycerine product obtained from an industrial process without substantial purification. Industrial glycerine can also be designated as “raw glycerine”, “raw glycerol” or “industrial glycerol”. Industrial glycerine contains more than about 70% of glycerol, preferably more than about 80%, less than 15% of water and impurities such as mineral salts and fatty acids. The concentration of mineral salts is less than 10%, preferably less than 5%. The concentration of fatty acids is less than 20%, preferably less than 5%. Fatty acids most represented in the industrial glycerine are palmitic acid, stearic acid, oleic acid, linolenic acid, linoleic acid and arachidic acid.

Industrial processes from which industrial glycerine is obtained are, inter alia, manufacturing methods where fats and oils, particularly fats and oils of plant origin, are processed into industrial products such as detergent or lubricants. In such manufacturing methods, industrial glycerine is considered as a by-product.

In a particular embodiment, the industrial glycerine is a by-product from biodiesel production and comprises known impurities of glycerine obtained from biodiesel production, comprising about 80 to 85% of glycerol with salts, water and some other organic compounds such as fatty acids. Industrial glycerine obtained from biodiesel production has not been subjected to further purification steps.

The term “synthetic medium” means a culture medium comprising a chemically defined substrate upon which organisms are grown.

In the culture medium of the present invention, glycerol is advantageously the single source of carbon. Preferably, this culture medium does not contain any organic nitrogen source. Nitrogen is a naturally occurring element that is essential for growth and reproduction in both plants and animals. It is found in amino acids and in many other organic and inorganic compounds. “Organic nitrogen” means, according to the invention, a nitrogen comprising organic compound obtained from living organisms. Usual sources of organic nitrogen for bacterial culture comprise yeast extract.

In a preferred embodiment, the producing microorganism is a Clostridium strain, more preferably Clostridium acetobutylicum.

In another embodiment of the invention, the producing microorganism is a genetically modified bacteria.

The phrase “genetically modified bacteria” means that the strain has been transformed in the aim to change its genetic characteristics. Endogenous genes can be attenuated, deleted, or over-expressed. Exogenous genes can be introduced, carried by a plasmid, or integrated into the genome of the strain, to be expressed into the cell.

The term “plasmid” or “vector” as used herein refers to an extra chromosomal element often carrying genes which are not part of the central metabolism of the cell, and usually in the form of circular double-stranded DNA molecules.

In another embodiment of the invention, the method is characterized in that the producing microorganism has a glycerol dehydratase activity that is independent of the presence of coenzyme B12 or one of its precursors, and that is derived from Clostridium butyricum.

In particular, the producing microorganism presents an increased flux of 1,3-propanediol production by introducing extra copies of the 1,3-propanediol operon from C. butyricum, (coding for enzymes involved in the vitamin B12-independent 1,3-propanediol pathway) either over-expressed by a plasmid or integrated into the chromosome of the microorganism. For example the pSPD5 plasmid can be used for an over-expression of the 1,3-propanediol operon.

Method for directing the glycerol metabolism towards production of 1,3-propanediol are known in the art (see for instance WO2006/128381, González-Pajuelo & al. 2006).

In another embodiment of the invention, the producing microorganism is a microorganism that has been previously adapted to grow in the presence of a high concentration of industrial glycerine. Said “adaptation” of the producing microorganism is obtained by culturing the microorganism on a culture medium comprising high concentration of industrial glycerine at a low dilution rate, and selecting the adapted microorganism able to grow on the culture medium having high concentration of industrial glycerine.

The present invention is also related to a method for the modification of a microorganism into a microorganism adapted to grow in the presence of a high concentration of industrial glycerine.

Several “adaptation processes” may be chosen by the person skilled in the art to transform the producing microorganism into a producing microorganism allowed to grow in the presence of a high concentration of industrial glycerine.

According to the present invention, the modification of a microorganism into a microorganism adapted to grow in the presence of a high concentration of industrial glycerine comprises culturing the microorganism on a culture medium comprising a high concentration of industrial glycerine at a low dilution rate and selecting the adapted microorganism able to grow on the culture medium having a high concentration of industrial glycerine.

The microorganism is advantageously cultured at a low dilution rate over a period ranging from 24 hours to 10 days, preferably more than 2 days, more preferably about 8 days.

The dilution rate is generally comprised between 0.005 and 0.1 h⁻¹, preferably between 0.005 and 0.02 h⁻¹. The dilution rate can be changed during the adaptation method, eventually with a first step comprised between 0.005 and 0.02 h⁻¹ and a second step where the dilution rate is increased up to 0.1 h⁻¹, more preferably 0.06 h⁻¹. When the dilution rate is modified during the adaptation method, dilution rate between 0.005 and 0.02 h⁻¹ are called “low dilution rate” while dilution rate between 0.02 and 0.1 h⁻¹ are common dilution rate.

The invention is also related to a microorganism adapted to grow in the presence of a high concentration of industrial glycerine susceptible to be obtained by the method as described above.

The microorganism adapted with the method of the present invention is preferably a producing microorganism being further adapted to grow in the presence of a high concentration of industrial glycerine.

Said adapted microorganism can also be a microorganism first adapted to grow in the presence of a high concentration of industrial glycerine further modified to have its glycerol metabolism directed to 1,3-propanediol production.

The “adapted microorganism” according to the invention is a producing microorganism adapted to grow in the presence of a high concentration of industrial glycerine and has two essential characteristics:
its glycerol metabolism is directed to 1,3-propanediol production, and
it is allowed to grow in the presence of a high concentration of industrial glycerine.

In a specific embodiment of the invention, the C. acetobutylicum DG1 pSPD5 strain is cultivated in continuous culture using a fed medium containing 105 g.l⁻¹ of raw glycerol from the biodiesel production, at a low dilution rate comprised between 0.005 and 0.02 h⁻¹, preferably 0.02 h⁻¹. Over a period of maximum 10 days, preferably between 5 and 8 days, the strain is adapted to the high glycerine concentration present in the fed medium, and the dilution rate can be increased up to 0.1 h⁻¹, preferably up to 0.06 h⁻¹ (see example 2).

The gradual increase of the dilution rate can be done between the end of the batch phase and 10 days, preferentially after 5 days, between 5 and 8 days, about 7 days, resulting in an improved productivity of the continuous culture (see example 3).

Advantageously, after the adaptation phase, the glycerol concentration in the fed medium can be increased up to 120 g.l⁻¹. However, direct attempt to adapt the strain to with 120 g.l⁻¹ of industrial glycerol is not possible, even at a dilution rate of 0.02 h⁻¹ as for a glycerol concentration of 105 g.l⁻¹, again demonstrating that the strain is not able to grow in the presence of a high glycerine concentration in the fed medium without prior adaptation.

The method of the invention, in its different embodiments (use of genetically modified microorganism and/or use of medium without additional organic nitrogen source), leads to production of 1,3-propanediol with a yield comprised between 0.4 and 0.6 g.g⁻¹ and a productivity comprised between 1.8 and 3.5 g.l⁻¹.h⁻¹ for a dilution rate comprised between 0.05 and 0.6 g.h⁻¹. Preferably the yield is comprised between 0.5 and 0.56 g.g⁻¹ and the productivity between 2 and 2.9 g.l⁻¹.h⁻¹.

In a specific aspect of the invention, the produced 1,3-propanediol is furthermore purified.

Continuous fermentation processes are known to the person skilled in the art.

The fermentation process is generally conducted in reactors with an inorganic culture medium of known defined composition adapted to the bacteria used, containing at least glycerine, a by-product from biodiesel production containing glycerol, and if necessary a co-substrate for the production of the metabolite.

This method of the invention is preferably realized in a continuous process. The man skilled in the art knows how to manage each of these experimental conditions, and to define the culture conditions for the microorganisms according to the invention. In particular clostridia are fermented at a temperature between 20° C. and 60° C., preferentially between 25° C. and 40° C. for C. acetobutilicum.

Methods for recovering and eventually purifying 1,3-propanediol from a fermentation medium are known to the skilled person. 1,3-propanediol may be isolated by distillation. In most embodiment, 1,3-propanediol is distilled from the fermentation medium with a by-product, such as acetate, and then further purified by known methods.

The present invention also concerns biosourced 1,3-propanediol obtained according to the method described above.

The present invention also concerns biosourced 1,3-propanediol characterized by its isotope ratios. Analysis of isotope ratios of hydrogen (D/H) and carbon (¹³C/¹²C) provides authenticity indicators for specific products such as honey (Grenier-Loustalot et al., 2006) or wine (Guillou et al, 2001). The ¹³C/¹²C isotope ratio provides indications for source discrimination and reflects the biosynthetic metabolic pathway of the specific product and the used raw material. Indeed difference could be made between C3 plants using Calvin-Benson photosynthetic cycle and C4 plants using Hatch-Slack photosynthetic cycle. The patent application WO 01/11070 describes a ¹³C/¹²C isotope ratio of biosourced 1,3-propanediol produced from glucose between −10.9 and −15.4; a ¹³C/¹²C isotope ratio of biosourced 1,3-propanediol produced from glycerol between −22.41 and −22.60 while ¹³C/¹²C isotope ratio of chemically produced 1,3-propanediol is comprised between −17.95 and −18,33.

According to the present invention, 1,3-propanediol D/H ratio (noted δD), ¹³C/¹²C ratio (noted δ¹³C), ¹⁸O/¹⁶O ratio (noted δ¹⁸O) were determined by mass spectrometry after combustion. The isotope ¹³C/¹²C ratio was calculated as δ per mill (%) with reference to the international standard (PDB=Pee Dee Belemite). The isotope ¹⁸O/¹⁶O ratio was calculated as δ per mill (%) with reference to the international standard Mean Ocean Water (SMOW). The isotope D/H ratio was calculated as ppm compare to the international standard Mean Ocean Water (SMOW).

Even though D/H ratio and ¹³C/¹²C ratios are well known for the characterisation of a given product, ¹⁸O/¹⁶O ratio is mainly used for water analysis as a paleoclimatic indicator.

Comparison between different types of 1,3-propanediol shows that δD is not discriminatory. δD for 1,3-propanediol biosourced from glycerol (object of the invention) is between 147.38 ppm and 145.84 ppm whereas is 145 ppm for 1,3-propanediol biosourced from glucose and between 150.19 ppm and 139.37 ppm for chemical 1,3-propanediol.

The invention concerns the biosourced 1,3-propanediol susceptible to be obtained according to the method described above, characterized by δ¹³C isotopic values lower than −34%, preferably lower than −35% and more preferably comprised between −35.05% and −36.09%.

The invention also concerns the biosourced 1,3-propanediol susceptible to be obtained according to the method described above, characterized by δ¹⁸O isotopic values comprised between 21.5% and 0.5%, preferably between 21.9% and 15% and more preferably comprised between 21.9% and 17.34%.

The invention also concerns a biosourced 1,3-propanediol characterized by one of the following characteristics or a combination thereof:
¹³C and ¹⁸O isotopic values selected among δ¹³C lower than −34% and δ¹⁸O between 21.9% and 0.5%,
preferably δ¹³C is lower than −35% and δ¹⁸O is between 21.9% and 0.5%,
more preferably δ¹³C is between −35.05% and −36.09% and δ¹⁸O is between 21.9% and 17.34% (See example 4).
   In one embodiment of the invention the biosourced 1,3-propanediol is characterized by a ¹⁸O/¹³C isotopic ratio value comprised between −2 and 0; preferably between −1 and −0.2 and more preferably between −0.65 and −0.4.

Preferably, biosourced 1,3-propanediol characterized by the previous isotope ratios is obtained by fermentation based on glycerol as raw material. More preferably, biosourced 1,3-propanediol characterized by the previous isotope ratios is obtained from the method of the invention for the production of 1,3-propanediol.

The present invention also concerns the use of biosourced 1,3-propanediol obtained by the method of the invention, or as defined above, as extender chain in thermoplastic polyurethanes, as monomers in polytrimethylene terephtalate or as component in cosmetic formulations. Biosourced 1,3-propanediol may be used in all known applications of chemical 1,3-propanediol. The man skilled in the art knows how to obtain these final products from 1,3-propanediol.

The present invention concerns methods of preparation of thermoplastic polyurethanes using as extender chain a biosourced 1,3-propanediol according to the invention. It also concerns methods of preparation of cosmetic compositions containing biosourced 1,3-propanediol according to the invention. It also concerns methods of synthesis of polytrimethylene terephtalate using biosourced 1,3-propanediol according to the invention as monomer.

### DRAWINGS

FIG. 1: PDO δ¹³C and δ¹⁸O in % of the international standard: PDO1: PDO produced according to the described process; PDO2: PDO produced from glucose as a substrate; PDO3: PDO produced by chemical process.

FIG. 2: δ¹⁸O/δ¹³C ratio of PDO: PDO1: PDO produced according to the described process; PDO2: PDO produced from glucose as a substrate; PDO3: PDO produced by chemical process.

### EXAMPLES

### Example 1

The synthetic media used for Clostridium batch cultivations contained per litre of de-ionized water: glycerol, 30 g; KH2PO4, 0.5; K2HPO4, 0.5 g; MgSO4, 7H2O, 0.2 g; CoCl2 6H2O, 0.01 g, H2SO4, 0.1 ml; NH4Cl, 1.5 g, biotin, 0.16 mg; p-amino benzoic acid, 32 mg and FeSO4, 7H2O, 0.028 g. The pH of the medium was adjusted to 6.3 with NH4OH 3N. For batch cultivation commercial glycerol purchased from Sigma (purity 99.5%) was used. The feed medium for continuous cultures contained par litre of tap water: raw glycerol, 105 g; KH2PO4, 0.5; K2HPO4, 0.5 g; MgSO4, 7H2O, 0.2 g; CoCl2 6H2O, 0.026 g; NH4Cl, 1.5 g, biotin, 0.16 mg; p-amino benzoic acid, 32 mg; FeSO4, 7H2O, 0.04 g, anti-foam, 0.05 ml; ZnSO4, 7H2O, 8 mg; CuCl2, 2H2O, 4 mg; MnSO4, H2O, 40 mg, H3BO3, 2 mg; Na2MoO4, 2H2O, 0.8 mg. Medium pH was not adjusted in this case. Raw glycerol resulting from the trans-esterification process of biodiesel production was supplied by SAIPOL (Le Meriot, France) and contained 83% glycerol (w/w).

Continuous cultures were performed in a 2 L bioreactor Tryton (Pierre Guerin, France) with a working volume of 1000 ml. The culture volume was kept constant at 1000 ml by automatic regulation of the culture level. Cultures were stirred at 200 RPM, at a temperature of 35° C. and pH was maintained at 6.5 by automatic addition of NH4OH 3N. The Oxido-Reductive Power of the culture medium expressed in mV, was controlled and recorded during the experiment. To create anaerobic conditions, the sterilized medium in the vessel was flushed with sterile O2-free nitrogen for one hour at 60° C. and flushed again until 35° C. was reached. The bioreactor gas outlet was protected form the oxygen by a pyrogallol arrangement (Vasconcelos et al., 1994). After sterilisation the feed medium was also flushed with sterile O2-free nitrogen until room temperature was attained and maintained under nitrogen pressure at 200 mbar to avoid O2 entry.

Cell concentration was measured turbidimetrically at 620 nm and correlated with cell dry weight evaluated directly. Glycerol, 1,3-propanediol, ethanol, butanol, acetic and butyric acids and others trace levels acids concentrations were measured by HPLC analysis. Separation was performed on a Biorad Aminex HPX-87H column and detection was achieved by refractive index. Operating conditions were as follows: mobile phase sulphuric acid 0.5 mM; flow rate 0.5 ml/min, temperature, 25° C.

A growing culture in 100 ml flasks on synthetic medium (the same as the batch culture medium described above but with the addition of acetic acid, 2.2 g.l⁻¹ and MOPS, 23.03 g.l⁻¹) taken at the end of exponential growth phase was used as inoculums (5% v/v). The cultures were first grown batch-wise. At the early exponential growth phase a pulse of commercial glycerol was added: the pulse is defined by an addition of synthetic medium (the same as described for batch culture) with commercial glycerol 120 g.l⁻¹ at a flow rate of 50 ml.h⁻¹ during 3 hours (i.e. an addition of 18 g of glycerol). Then the growth continued batch-wise and before the end of the exponential growth phase the continuous feeding started with a dilution rate of 0.06 h⁻¹ and a feed medium containing 105 g.l⁻¹ of raw glycerol.

TABLE 1
Continuous culture of C. acetobutylicum DG1 (pSPD5) on raw
glycerol at 105 g · l⁻¹ (D = 0.06 h − 1, pH 6.5 ant T° C. = 35° C.).
The given values represent an average of 7 points in
steady state condition obtained after 3 volume changes.

| | Average and standard |
|---|---|
| | deviation |
| Feed glycerol (g · l⁻¹) | 106.1 +/− 0.00 |
| 1,3-propanediol (g · l⁻¹) | 32.68 +/− 1.16 |
| Y_{1,3-PDO} (g · g⁻¹) | 0.50 +/−0.02 |
| Q_{1,3PDO} (g · l⁻¹ · h⁻¹) | 2.00 +/− 0.09 |
| Dilution rate (h⁻¹) | 0.061 +/− 0.003 |
| Residual glycerol (g · l⁻¹) | 36.38 +/− 1.88 |
| Biomass (g · l⁻¹) | 1.83 +/− 0.09 |
| Acetic acid (g · l⁻¹) | 0.99 +/− 0.07 |
| Butyric acid (g · l⁻¹) | 7.65 +/− 0.44 |
| Carbon recovery (%) | 96.9 +/− 2.5 |

Y_{1,3-PDO}: PDO yield (g/g of glycerol consumed)
Q_{1,3PDO}: PDO volumetric productivity
For the carbon recovery calculation, carbon dioxide concentration was estimated from end-products concentrations.

### Example 2

The synthetic media used were the same as described in example 1. Pre-culture, Inoculation and batch-wise growth were performed in the same conditions as described above.

The continuous feeding started with a dilution rate of 0.02 h⁻¹ and a feed medium containing 105 g.l⁻¹ of raw glycerol. After few days in these conditions (i.e. 8-10 days after inoculation of the bioreactor corresponding to 3 volumes changes at least) the dilution rate was increased from 0.02 h⁻¹ to 0.05 h⁻¹ according to the following scheme: i) increase of 0.01 h⁻¹ unit in 48 hours and ii) resting step of 24-48 hours, repeated 3 times. Stability of the culture was followed by products analysis using the HPLC protocol previously described. Notably, we waited for residual glycerol to be as low as possible to make a final increased of the dilution rate to 0.06 h⁻¹ in 48 hours.

TABLE 2
Continuous culture of C. acetobutylicum DG1
(pSPD5) on raw glycerol at 105 g · l⁻¹ (D = 0.05 h − 1 and
0.06 h − 1, pH 6.5 ant T° C. = 35° C.). The given values
represent an average of 3 or 4 points in steady state condition.

| | Average and | Average and |
|---|---|---|
| | standard deviation | standard deviation |
| | for D = 0.05 h⁻¹ | for D = 0.06 h⁻¹ |
| Feed glycerol (g · l⁻¹) | 108.36 +/− 0.00 | 110.01 +/− 0.00 |
| 1,3-propanediol (g · l⁻¹) | 51.92 +/− 0.29 | 50.95 +/− 0.52 |
| Y_{1,3-PDO} (g · g⁻¹) | 0.53 +/− 0.00 | 0.51 +/− 0.01 |
| Q_{1,3PDO} (g · l⁻¹ · h⁻¹) | 2.68 +/− 0.04 | 2.87 +/− 0.02 |
| Dilution rate (h⁻¹) | 0.052 +/− 0.001 | 0.056 +/− 0.000 |
| Residual glycerol (g · l⁻¹) | 3.66 +/− 0.40 | 4.51 +/− 0.33 |
| Biomass (g · l⁻¹) | 1.94 +/− 0.07 | DO |
| Acetic acid (g · l⁻¹) | 3.27 +/− 0.11 | 2.67 +/− 0.06 |
| Butyric acid (g · l⁻¹) | 10.75 +/− 0.44 | 10.97 +/− 0.21 |
| Carbon recovery (%) | 97.4 +/− 1.4 | 95.1 +/− 1.2 |

Y_{1,3-PDO}: PDO yield (g/g of glycerol consumed)
Q_{1,3PDO}: PDO volumetric productivity
For the carbon recovery calculation, carbon dioxide concentration was estimated from end-products concentrations.

Another culture performed in the same conditions led to the same results.

### Example 3

The synthetic media used were the same as described in example 1, except that the feed medium contained 120 g.l⁻¹ of raw glycerol. Pre-culture, Inoculation and batch-wise growth were performed in the same conditions as described above.

A) The continuous feeding started with a dilution rate of 0.02 h⁻¹ and a feed medium containing directly 120 g.l⁻¹ of raw glycerol.

TABLE 3
Continuous culture of C. acetobutylicum DG1
(pSPD5) on raw glycerol at 120 g · l⁻¹ (D = 0.02 h⁻¹,
pH 6.5 ant T° C. = 35° C.). The given values
represent an average of 7 points in steady state
condition obtained after 3 volume changes.

| | Average and |
|---|---|
| | standard |
| | deviation |
| Feed glycerol (g · l⁻¹) | 123.67 +/− 0.00 |
| 1,3-propanediol (g · l⁻¹) | 42.13 +/− 1.47 |
| Y_{1,3-PDO} (g · g⁻¹) | 0.50 +/− 0.01 |
| Q_{1,3PDO} (g · l⁻¹ · h⁻¹) | 1.01 +/− 0.05 |
| Dilution rate (h⁻¹) | 0.023 +/− 0.001 |
| Residual glycerol (g · l⁻¹) | 30.87 +/− 2.62 |
| Biomass (g · l⁻¹) | 1.19 +/− 0.09 |
| Acetic acid (g · l⁻¹) | 2.36 +/− 0.18 |
| Butyric acid (g · l⁻¹) | 9.32 +/− 0.45 |
| Carbon recovery (%) | 94.2 +/− 1.4 |

Y_{1,3-PDO}: PDO yield (g/g of glycerol consumed)
Q_{1,3PDO}: PDO volumetric productivity
For the carbon recovery calculation, carbon dioxide concentration was estimated from end-products concentrations.

B) The continuous feeding started with a dilution rate of 0.02 h⁻¹ and a feed medium containing first 105 g.l⁻¹ of raw glycerol. After few days in these conditions (corresponding to 3 volumes changes at least), the dilution rate was increased from 0.02 h⁻¹ to 0.06 h⁻¹ according to the scheme described previously. Then the feeding was switched to a dilution rate of 0.05 h⁻¹ and a feed medium containing 120 g.l⁻¹ of raw glycerol.

TABLE 4
Continuous culture of C. acetobutylicum DG1 (pSPD5)
on raw glycerol at 120 g · l⁻¹ (D = 0.05 h⁻¹, pH 6.5
ant T° C. = 35° C.) after feeding at 105 g · l⁻¹ raw glycerol
at D = 0.06 h⁻¹. The given values represent an average
of 3 points obtained after at least 20 volume changes.

| | Average and |
|---|---|
| | standard |
| | deviation |
| Feed glycerol (g · l⁻¹) | 123.68 +/− 0.00 |
| 1,3-propanediol (g · l⁻¹) | 53.53 +/− 0.59 |
| Y_{1,3-PDO} (g · g⁻¹) | 0.53 +/− 0.02 |
| Q_{1,3PDO} (g · l⁻¹ · h⁻¹) | 2.86 +/− 0.02 |
| Dilution rate (h⁻¹) | 0.053 +/− 0.000 |
| Residual glycerol (g · l⁻¹) | 15.58 +/− 3.56 |
| Biomass (g · l⁻¹) | 1.14 +/− 0.18 |
| Acetic acid (g · l⁻¹) | 4.30 +/− 0.10 |
| Butyric acid (g · l⁻¹) | 9.29 +/− 0.17 |
| Carbon recovery (%) | 94.8 +/− 2.3 |

Y_{1,3-PDO}: PDO yield (g/g of glycerol consumed)
Q_{1,3PDO}: PDO volumetric productivity
For the carbon recovery calculation, carbon dioxide concentration was estimated from end-products concentrations.

### Example 4

### Methods for Calculation of the δ¹³C and δ¹⁸O Values

Levels of ¹³C and ¹⁸O are by convention expressed as relative values. Normally they are expressed in the form of a percentage (δ) when compared to two internationals references.

For the ¹³C, the reference is the “Vienna.PD belemnite” which is a fossil carbonate from which the ¹³C value is known.

The formula for the calculation of the δ is as follow:

$$δ {}^{13} C ( {}_{}^{o}{\text{/}}{}_{oo}^{} ) = \frac{{( {}^{13} C / {}^{12} C )}_{spl} - {( {}^{13} C / {}^{12} C )}_{ref}}{{( {}^{13} C / {}^{12} C )}_{ref}} \times 1000$$

For the ¹⁸O the reference is the “Standard Mean Ocean Water” (SMOW) with a known ¹⁸O value. The formula is identical to the ¹³C one, with the ¹⁸O reference value.

### Used Materials and Sample Preparation

### ¹³C

The ¹³C/¹²C isotopic ratio of PDO was calculated based on the carbon dioxide specimen measured experimentally. For this purpose the samples were burned in an elemental analyzer and the carbon dioxide obtained was injected into a mass spectrometer (Finnigan MAT DELTA) coupled to an elemental analyzer (CARLO ERBA NA 2100) for the determination of the isotopic ratio. In this way the 44, 45 and 46 masses of carbon dioxide were separated and quantified. The ¹³C/¹²C isotopic ratio was then calculated on the delta per thousand scale by comparing the results with the one of the working reference (Glutamic acid) which is calibrated versus the international standard beforehand.

### ¹⁸O

The measurement of the ¹⁸O/¹⁶O isotopic ratio was done in a continuous flux of an organic compound. For this purpose the samples were burned by pyrolysis at the level of a microanalyzer oven and the carbon monoxide produced was sent into the mass spectrometer (OPTIMA coupled with an elemental analyzer Fisons NA1500 2 series, or, a mass spectrometer Delta V coupled to an elemental analyzer TC/EA from Thermoelectron). Different isotopes of 28, 29, 30 were determined in the carbon monoxide produced by pyrolysis. The ¹⁸O/¹⁶O isotopic ratio was then calculated on the delta per thousand scale by comparing the results with SMOW.

### Results

For PDO produced from raw glycerol we obtained an average δ¹³C value of −35.57±0.52%, PDO produced from glucose has an average δ¹³C value of −12.6% and PDO produced chemically has an average δ¹³C value of −30.05±5.02%.

PDO produced from raw glycerol has an average δ180 value of 19.76±2.42%, PDO produced from glucose has an average δ¹⁸O value of 22.0% and PDO produced chemically has an average δ¹⁸O value of −0.80±1.27%.

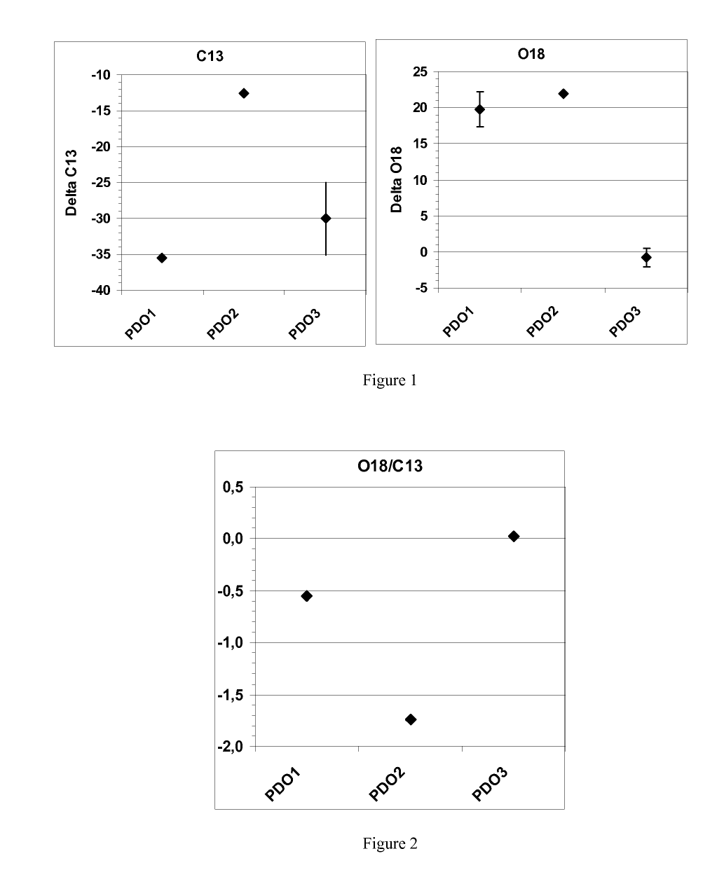

Thus the measurement of δ¹³C allows distinguishing PDO produced by a fermentation process from diverse raw material. The δ¹³C of chemically produced PDO shows high variability (−30.05±5.02) and is only slightly higher than the δ¹³C of PDO produced from glycerol. The δ¹⁸O of the chemically produced PDO has a very low value (−0.8±1.27) which makes it possible to distinguish between a chemically produced PDO and a PDO from biological origin, either glucose or glycerol based (FIG. 1). The δ¹⁸O/δ¹³C value clearly allows identifying the different PDOs (FIG. 2) with a δ¹⁸O/δ¹³C ratio of −0.56 for glycerol based PDO, −1.75 for glucose based PDO and 0.03 for chemically produced PDO. In conclusion, PDO produced from glycerol can be identified via the measurements of δ¹³C and δ¹⁸O followed by the determination of the δ¹⁸O/δ¹³C.

### REFERENCES

1. Cotte J F, Casabianca H, Lhéritier J, Perrucchietti C, Sanglar C, Waton H and Grenier-Loustalot M F. 2007. Study and validity of 13C stable carbon isotopic ratio analysis by mass spectrometry and 2H site specific natural isotopic fractionation by nuclear magnetic resonance isotopic measurements to characterize and control the authenticity of honey. Analytica Chimica Acta 582: 125-136.
2. González-Pajuelo M, Meynial-Salles I, Mendes F, Andrade J C, Vasconcelos I, and Soucaille P. 2005. Metabolic engineering of Clostridium acetobutylicum for the industrial production of 1,3-propanediol from glycerol. Metabolic Engineering 7: 329-336.
3. González-Pajuelo M, Meynial-Salles I, Mendes F, Soucaille P, and Vasconcelos I. 2006. Microbial conversion of a natural producer, Clostridium butyricum VPI 3266, and an engineered strain, Clostridium acetobutylicum DG (pSPD5). Applied and Environmental Microbiology, 72: 96-101.
4. Guillou C, Jamin E, Martin G J, Reniero F, Wittkowski R and Wood R. 2001. Analyses isotopiques du vin et des produits dérivés duraisin. Bulletin de I'O.I.V: 839-840.
5. Nakas J P, Schaedle M, Parkinson C M, Coonley C E, and Tanenbaum S W. 1983. System development for linked-fermentation products of solvents from algal biomass. Applied and Environmental Microbiology 46: 1017-1023.
6. Papanikolaou S, Ruiz-Sanchez P, Pariset B, Blanchard F and Fick M. 2000. High production of 1,3-propanediol from industrial glycerol by a newly isolated Clostridium butyricum strain. Journal of Biotechnology. 77: 191-2008.
7. WO2006/128381. Method for preparing 1,3-propanediol by using glycerine as the by-product of the biological diesel oil.
8. WO01/11070. 1,3-propanediol and polymer derivatives from a fermentable carbon source.

1-22. (canceled)

23. A method for the production of 1,3-propanediol in a continuous fermentation process of glycerine, comprising culturing a producing microorganism on a culture medium, said producing microorganism allowing the conversion of glycerol into 1,3-propanediol, and recovering the 1,3-propanediol, wherein the culture medium comprises a high concentration of industrial glycerine, said industrial glycerine comprising glycerol, and wherein the producing microorganism is a microorganism previously adapted to grow in the presence of a high concentration of industrial glycerine.

24. The method according to claim 23, wherein in the culture medium the glycerol is present at a concentration comprised from 90 to 120 g/L.

25. The method according to claim 24, wherein in the culture medium the glycerol is present at a concentration of about 105 g/L.

26. The method according to claim 23, wherein the industrial glycerine is a by-product from biodiesel production.

27. The method according to claim 23, wherein the culture medium is a synthetic medium without addition of organic nitrogen source.

28. The method according to claim 27, wherein the culture medium is a synthetic medium without addition of yeast extract.

29. The method according to claim 23, wherein the producing microorganism is selected from the group consisting of members of the genus Clostridium.

30. The method according to claim 29, wherein the producing microorganism comprises Clostridium acetobutylicum.

31. The method according to claim 23, wherein the producing microorganism is a genetically modified bacteria.

32. The method according to claim 23, wherein the glycerol dehydratase activity in the producing microorganism is independent of the presence of coenzyme B 12 or a precursor thereof and is derived from Clostridium butyricum.

33. The method according to claim 23, wherein the producing microorganism is adapted into a producing microorganism by culturing the microorganism on a culture medium comprising high concentration of industrial glycerine at a low dilution rate and selecting the adapted microorganism able to grow on the culture medium having high concentration of industrial glycerine.

34. The method according to claim 23, wherein the 1,3-propanediol is further purified.

35. A method for the modification of a microorganism into a microorganism adapted to grow in the presence of a high concentration of industrial glycerine, comprising culturing the microorganism on a culture medium comprising high concentration of industrial glycerine at a low dilution rate and selecting the adapted microorganism able to grow on the culture medium having high concentration of industrial glycerine.

36. The method of claim 35, wherein the microorganism is cultured at low dilution rate over a period ranging from 24 hours to 10 days.

37. The method of claim 35, wherein the dilution rate is comprised from 0.005 to 0.1 h⁻¹.

38. A microorganism adapted to grow in the presence of a high concentration of industrial glycerine, susceptible to be obtained by a method comprising culturing the microorganism on a culture medium comprising high concentration of industrial glycerine at a low dilution rate and selecting the adapted microorganism able to grow on the culture medium having high concentration of industrial glycerine.

39. Biosourced 1,3-propanediol obtained according to the method of claim 23.

40. Biosourced 1,3-propanediol capable of being obtained according to the method of claim 23, and having a δ¹³C isotopic value lower than −34%.

41. Biosourced 1,3-propanediol capable of being obtained according to the method of claim 23, and having a δ¹³C isotopic value less than −35%.

42. Biosourced 1,3-propanediol capable of being obtained according to the method of claim 23, and having a δ¹³C isotopic value comprised from −35.05% to −36.09%.

43. Biosourced 1,3-propanediol capable of being obtained according to the method of claim 23, and having a δ⁸⁰O isotopic value comprised from 21.5% to 0.5%.

44. Biosourced 1,3-propanediol capable of being obtained according to the method of claim 23, and having a δ¹⁸O isotopic value comprised from 21.9% to 15%.

45. Biosourced 1,3-propanediol capable of being obtained according to the method of claim 23, and having a δ¹⁸O isotopic value comprised from 21.9% to 17.34%.

46. Biosourced 1,3-propanediol capable of being obtained according to the method of claim 23, and having a combination of δ¹³C and δ¹⁸O isotopic values selected among the following values:
δ¹³C value is lower than −34% and δ¹⁸O from 21.5% to 0.5%;
δ¹³C value is lower than −35% and δ¹⁸O from 21.9% to 15% or
δ¹³C value is of −35.05% to −36.09% and δ¹⁸O from 21.9% to 17.34%.

47. Biosourced 1,3-propanediol capable of being obtained according to the method of claim 23, and having a ¹⁸O/¹³C isotopic ratio value comprised from −2 to 0.

48. Biosourced 1,3-propanediol capable of being obtained according to the method of claim 23, and having a ¹⁸O/¹³C isotopic ratio value comprised from −1 to −0.2.

49. Biosourced 1,3-propanediol capable of being obtained according to the method of claim 23, and having a ¹⁸O/¹³C isotopic ratio value comprised from −0.65 to −0.4.

50. An extender chain in thermoplastic polyurethane synthesis comprising of 1,3-propanediol according to claim 39.

51. A component for a cosmetic formulation comprising 1,3-propanediol according to claim 39.

52. A monomer for synthesis of polytrimethylene terephtalate comprising 1,3-propanediol according to claim 39.
